(19) United States
(12) Patent Application Publication
(10) Pub. No.: US 2022/0023124 A1
(43) Pub. Date: Jan. 27, 2022

(54) MULTISIDE TILT DEVICE

(71) Applicant: SECRETARY, DEPARTMENT OF BIOTECHNOLOGY, New Delhi (IN)

(72) Inventors: Asish MOHANDAS, Palakkad (IN); Karthik P.B., Mangalore (IN); Prashant JHA, New Delhi (IN); Sushma SAGAR, New Delhi (IN)

(21) Appl. No.: 17/297,699

(22) PCT Filed: Nov. 27, 2019
(86) PCT No.: PCT/IN2019/050869

§ 371 Date: May 27, 2021

(51) Int. Cl. A61G 7/057; A61G 7/018; A61B 5/00

(52) U.S. Cl. CPC A61G 7/05784; A61G 7/05776; A61G 7/0573; A61G 2203/34; A61B 5/6892; A61B 2562/0247; A61G 7/018

(57) ABSTRACT

The present subject matter discloses multiside tilt device (100) including a pressure sensing sheet (102) to detect the pressure exerted by the user's body in a pressure build-up area around the pressure sensor and generate a pressure signal indicating a pressure build-up at the pressure build-up area. The multiside tilt device (100) further includes a tilting unit (104) to tilt a portion of the user's body in response to the pressure signal by a predetermined angle. The tilting unit (104) comprises an inflatable base (106) having a first section (108-1) and a second section (108-2) pneumatically separated disposed adjacent to each other to be independently inflated or deflated. Each section (108) comprises one or more cells (112, 304) to be collectively inflated such that each cell (112, 304) is inflated to form an inflated structure having a peak in the center and a trough on either side.

### TECHNICAL FIELD

The present subject matter relates, in general, to a multiside tilt device, and in particular to a multiside tilt device for repositioning of a person with limited mobility.

### BACKGROUND

People with limited mobility, such as patients and elderly may be required to stay in bed for long duration of time. During bedrest, a patient may experience continuous pressure developed due to the weight of the body against the surface of the bed which may reduce blood supply to skin. Reduction in blood supply to skin may damage the skin and underlying tissue. Such a medical condition is known as pressure ulcers, also known as pressure sores or bedsores.

### BRIEF DESCRIPTION OF DRAWINGS

The detailed description is described with reference to the accompanying figures. It should be noted that the description and figures are merely examples of the present subject matter and are not meant to represent the subject matter itself.

FIG. 1A-1B illustrates a multiside tilt device, according to an example implementation of the present subject matter.

FIGS. 2A-2D illustrates different views of a pressure sensing sheet of the multiside tilt device, according to an example implementation of the present subject matter.

FIGS. 3A-3D illustrates different views of an inflatable base of the multiside tilt device, according to an example implementation of the present subject matter.

FIGS. 4A-4B illustrates a control unit of the multiside tilt device, according to an example implementation of the present subject matter.

FIGS. 5A-4K illustrates different operational stages of the multiside tilt device, according to an example implementation of the present subject matter.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Bedsore or pressure ulcers is a medical condition where a person may develop ulcers on certain areas of the skin owing to pressure build up because of lying in bed for prolonged time. In order to reduce the risk of acquiring bedsores, a person may be periodically laterally tilted to redistribute the pressure building at the skin. Thus, people suffering from pressure ulcers are usually advised to frequently reposition themselves on the bed to avoid stress on the skin. Conventionally, a caregiver may be required whenever the individual lying on the bed wishes to reposition himself.

With advent in technology, pressure relieving and redistributing devices have been developed to prevent pressure ulcers. Such devices include overlays, cushions, and seating supports, and work by reducing pressure, friction or shearing forces. Such conventional devices can be used for a single body part of the individual which is at risk and are not useful for preventing pressure ulcers on multiple body parts of the individual simultaneously. Further, such devices require manual intervention and may require an additional personnel to take care of the person.

Other conventional devices may include tilting mattresses divided into two sections that are inflated or deflated to tilt a person laterally. However, such tilting mattresses includes linear shaped sections which, even when tilted, remain in contact with the person's body. As a result, they may not be able to effectively relieve the pressure from bony region of the person's body. This leads to continuous pressure build-up and may gradually cause pressure sores. Further, since the bony region of the person's body always remains in contact with the mattress, moisture on the skin may cause the skin to lose its dry outer layer and may reduce the tolerance of the skin to pressure and shear. Such tilting mattresses may also not provide any assistance in tilting the person. Further, usually, a person may be tilted according to routine time frame. The frequency of turning and repositioning depends on the level of risk in the person. For example, a person may be tilted or repositioned every one-hour, two-hour or even once or twice a day making the person highly susceptible to pressure sores. Even if the person is repositioned frequently, the person or nurse may not be aware of the exact time after which the person must be repositioned, thereby introducing inefficiencies.

The present subject matter discloses example implementations of a multiside tilting device. In an example implementation of the present subject matter, the multiside tilting device includes a pressure sensing sheet to support a user in a resting position. For example, user may be any patient, elderly person or a user with limited mobility. The pressure sensing sheet includes a plurality of pressure sensors positioned in the pressure sensing sheet at predetermined areas, also referred to as pressure build-up area, that may support pressure sensitive body parts of the user, i.e., body parts that are most likely to acquire bed sore. Each pressure sensor may thus detect pressure exerted by the user's body in the pressure build-up area around the pressure sensor. The pressure sensors may generate a pressure signal to indicate a pressure build-up at the pressure build-up area.

The multiside tilt device further includes a tilting unit positioned beneath the pressure sensing sheet. The tilting unit comprises an inflatable base having a plurality of sections. In one embodiment, the inflatable base includes a first section and a second section disposed adjacent to each other on either side of a median axis of the tilting unit. In one example, in a resting position, the user's spinal curve may lie in region corresponding to the median axis. In one example, the first section may support a right side of the user's body and a second section may support a left side of the user's body. In another example, the first section may support a left side of the user's body and a second section may support a right side of the user's body. The first section and the second section may be pneumatically separated from each other to be independently inflated or deflated in response to the pressure signal. In one implementation, the first section may be inflated in response to the pressure signal generated by the pressure sensors placed in the pressure sensing sheet in a first region above the first section. The second section may be inflated in response to the pressure signal generated by the pressure sensors placed in the pressure sensing sheet in a second region above the second section.

In one example, each section may include one or more cells pneumatically connected to each other to be collectively inflated or deflated in response to the pressure signal. Each cell may be inflated to form an inflated structure having a peak in the center and a trough on either side. In one embodiment, a part of the user's body resting over the section, may be in contact with the peak of each cell while other parts of the user's body resting over the section, and corresponding to the trough, are not in contact with the pressure build-up area. In one example, the bony prominence areas may hang in the air while the other parts of the user's body may remain in contact with the inflatable base.

For example, owing to a pressure build up by a right side of the user's body, the pressure sensors lying in the first region, below the right side of the user's body, may generate pressure signals having a value above a threshold level. Based on the pressure signals, the one or more cells in the first section may get inflated such that user's body part lying over the peak of the one or more cells in the first section and the user's body part lying over the second section may remain in contact with the multiside tilting device. Whereas user's body part lying over the troughs and up till the spinal region may be lifted, thereby, not being in contact with the multiside tilting device, thus getting relief from pressure.

Thus, the multiside tilt device may eliminate the pressure build-up at the bony prominence areas, thereby, avoiding the condition of bed sores. Further, since each cell of the section is inflated to form a peak, a small portion of the body comes in contact with the inflatable base, thereby, lifting the user with minimal contact being still maintained to provide adequate support to the user. In one example, such small portion of the user's body may be areas where the pressure build-up may be minimum. Further, the area around the spinal cord of the user, corresponding to the trough region of the inflated cell, may be slightly lifted and not be in contact with the multiside tilt device, thereby, relieving pressure. This may further allow air movement across the lifted body parts. Further, since the user is adequately supported from the both the right and left edges of the body, by non-inflated section and the peak of the inflated section, the user can safely rest. Providing the support from both the sides instead of a single side further helps in ensuring that no extra pressure is exerted on the single side supporting the body. As a result, the body may not experience severe pressure build-up at any particular area. This further reduces the chances of acquiring bed sores.

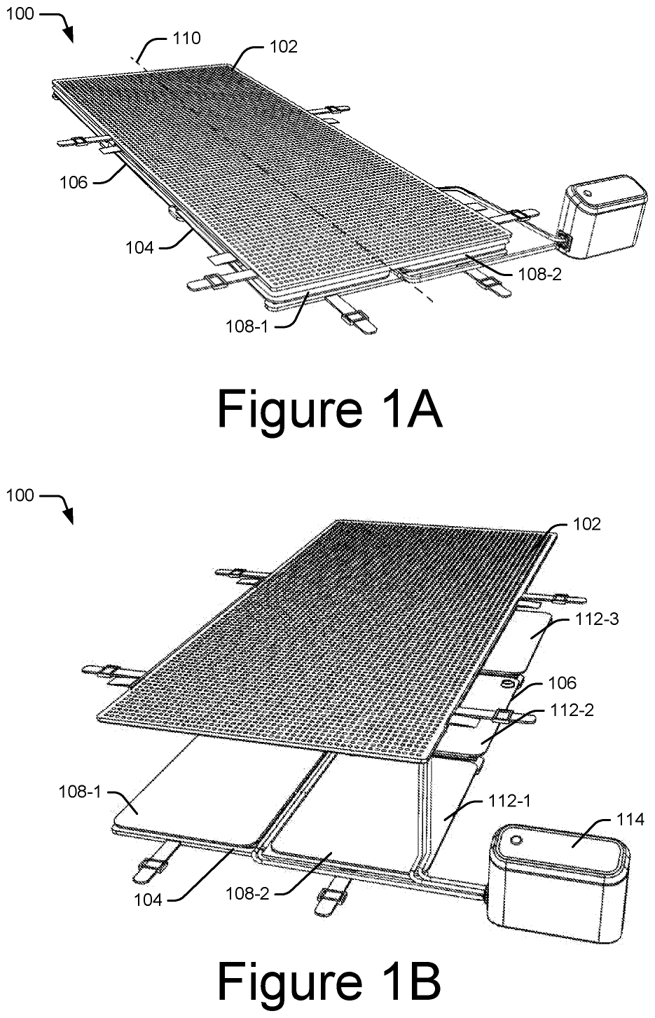

The present subject matter is further described with reference to FIGS. 1 to 4. It should be noted that the description and figures merely illustrate principles of the present subject matter. Various arrangements may be devised that, although not explicitly described or shown herein, encompass the principles of the present subject matter. Moreover, all statements herein reciting principles, aspects, and examples of the present subject matter, as well as specific examples thereof, are intended to encompass equivalents thereof.

FIG. 1a illustrates a multiside tilt device 100, in accordance with an implementation of the present subject matter. The multiside tilt device 100, hereinafter interchangeably referred to as device 100, may be used by user with limited mobility, such as patients and elderly, to reposition themselves while the user is resting on the device to prevent the development of bedsores/pressure ulcers/pressure sores. In one example, the multiside tilt device 100 may be retrofitted into any existing beds at home, hospital or any other healthcare settings on any type of resting surface. In one embodiment, the multiside tilt device 100 may be placed above a conventional mattress and below the patient.

In one example, the multiside tilt device 100 includes a pressure sensing sheet 102 to determine pressure exerted by the user's body at various areas of the body and generate a corresponding pressure signal in response to the determination. The pressure sensing sheet 102 may support a user in a resting position and a plurality of pressure sensors (not shown in this Figure) to detect a pressure exerted by the user's body in a pressure build-up area around the pressure sensor. Each pressure sensor may generate a pressure signal indicating a pressure build-up at the pressure build-up area around the pressure sensor. In one example, the plurality of pressure sensors may be positioned at predetermined areas which may correspond to and lie below those parts of user's body where a user is most likely to acquire bedsore, such as bony prominence areas of the user's body. The bony prominence areas may include, but are not limited to, occiput (back of head), scapula (shoulder blades), sacrum (tailbone area), and heels.

The multiside tilt device 100 may further include a tilting unit 104 to tilt the user's body and redistribute the pressure acting on the body of the user. The tilting unit 104 may be positioned beneath the pressure sensing sheet 102. The tilting unit 104 includes an inflatable base 106 having a first section 108-1 and a second section 108-2, hereinafter collectively referred to as sections 108 and individually as section 108. In one example, the first section 108-1 and the second section 108-2 may be disposed adjacent to each other on either side of a median axis 110 of the tilting unit 104. In one example, in the resting position, the user's spinal curve may lie in a region corresponding to the median axis 110. The median axis 110 may thus be presumed to be dividing the user's body into two symmetrical halves such that the plurality of sensors may be equally positioned in the predetermined areas.

In one example, the first section 108-1 and the second section 108-2 are pneumatically separated from each other to be independently inflated or deflated in response to the pressure signal. Each section 108 includes one or more cells pneumatically connected to each other to be collectively inflated or deflated in response to the pressure signal. In one example, each cell is inflated to form an inflated structure having a peak in the center and a trough on either side. When one of the section is inflated, a part of user's body resting over the section, may be in contact with the peak of each cell, while other parts of the user's body resting over the section, and corresponding to the trough, may not be in contact with the multiside tilt device to release pressure build-up.

FIG. 1b illustrates an exploded view of the device 100, in accordance with an embodiment of the present subject matter. As illustrated, the inflatable base 106 includes a first section 108-1 and a second section 108-2 disposed adjacent to each other. The first section 108-1 includes one or more cells (not shown in the figure) that may be collectively inflated or deflated in response to the pressure signal. Similarly, the second section 108-2 may also include one or more cells 112-1, 112-2, and 112-3 that may be collectively inflated or deflated in response to the pressure signal. The one or more cells 112-1, 112-2, and 112-3 corresponding to the second section 108-2 are hereinafter referred to as one or more cells 112 and individually as cell 112. The device 100 may further include a control unit 114 to control the inflation and deflation of the sections 108. In one example, the control unit 114 may be communicatively coupled to the pressure sensors to receive the pressure signal and analyze the pressure signals to determine if there is any pressure build-up under the user's body around the pressure sensors. The control unit 114 may accordingly inflate or deflate the sections 108.

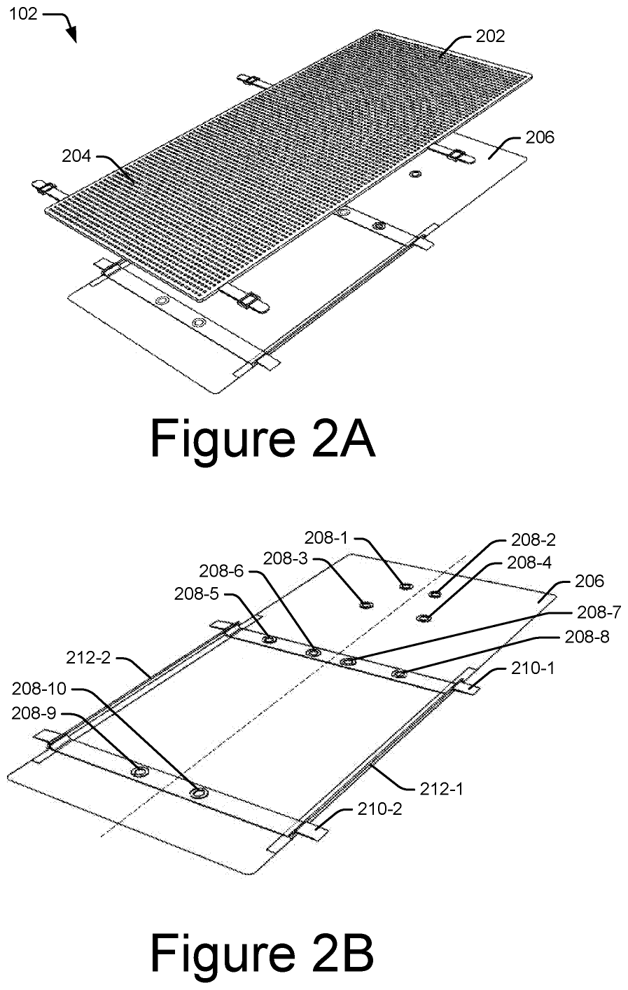

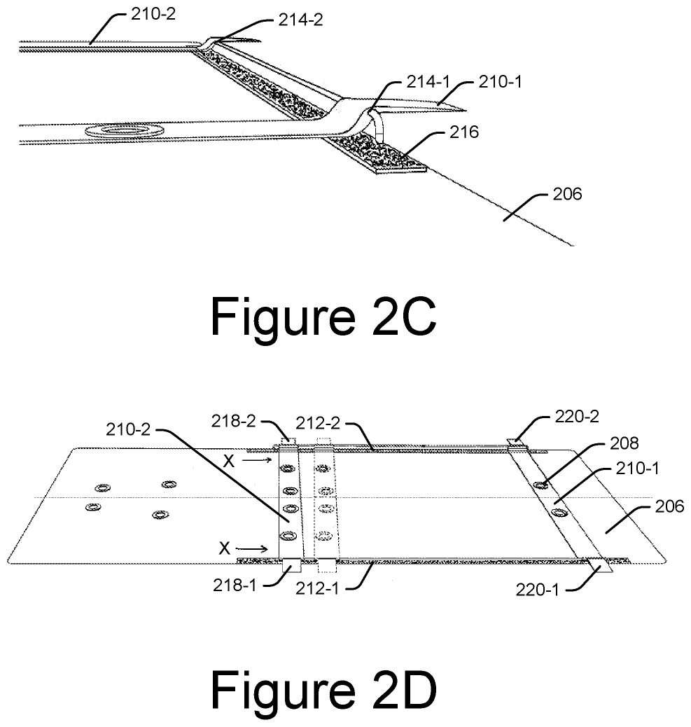

FIG. 2a illustrates a pressure sensing sheet 102 of the device 100, in accordance with an implementation of the present subject matter. In one example, the pressure sensing sheet 102 may continuously detect the pressure exerted by the user's body at different areas of pressure sensing sheet 102 and accordingly generate a pressure signal in response to the pressure detection. The pressure sensing sheet 102 may include a top layer 202 having perforations 204 for air passage to allow air to flow through the top layer. For the sake of brevity and clarity, the perforations 204 have been collectively labelled as 204 instead of being individually labelled. In one example, the perforations 204 may be artificially created, for example, by laser cutting. In another example, the material used for making the pressure sensing sheet 102 may have naturally occurring perforations. In one example, the top layer 202 may be made up of a material textured like cloth which may be compatible with skin, non-permeable to liquids, and permeable to air. The air coming out of the perforations may dry the upper layer of the user's skin to help prevent moisture build-up on the pressure sensing sheet 102 and the user's body. As a result, the top layer 202 may provide a low air loss surface, thereby reducing the probability of developing pressure ulcers/bedsores.

The pressure sensing sheet 102 may further include a bottom layer 206 coupled to the top layer 202 to form a sealed sheet. In one example, the top layer 202 and the bottom layer 206 may be sealed throughout their perimeter by means of stitching, heat sealing, HF welding, RF welding, any similar process or a combination thereof. The bottom layer 206 may be made up of, but not limited to, a fabric, non-permeable material or a combination thereof.

FIG. 2b illustrates the bottom layer 206 of the pressure sensing sheet 102, in accordance with an implementation of the present subject matter. In one example, the bottom layer 206 may be made up of a material selected from the group comprising a fabric, non-permeable polymeric material, and a combination thereof. The bottom layer 206 may include a plurality of pressure sensors 208-1, 208-2, 208-3, 208-4, 208-5, 208-7, 208-8, 208-9, and 208-10, hereinafter referred to as plurality of pressure sensors 208 and individually as pressure sensor 208. In one example, the pressure sensors 208 may be movably positioned at different areas of the bottom layer 206. In one example, the pressure sensor may be any one of pressure measuring units, sensors, piezoelectric sensors, thin film pressure sensors or a combination thereof. The pressure sensors 208 may be positioned at areas that are most likely to develop pressure sores, such as the bony prominence areas. The bony prominence areas may include, but are not limited to, occiput (back of head), scapula (shoulder blades), sacrum (tailbone area) and heels. In one embodiment, one or more pressure sensors out of the plurality of pressure may be fixed to a certain position while other pressure sensors out of the plurality of pressure sensors may be adjustable according to a height of the user's body. In one example, one or more of the plurality of sensors 208 positioned near the head region and the shoulder region may be fixed and one or more of the plurality of sensors 208 corresponding to heel area and sacrum area may be movably positioned on a movable part of the pressure sensing sheet 102 to allow adjustment of the pressure sensors 208 in accordance to a body structure, such as height of the user. As a result, the pressure sensors 208 corresponding to the heel area and the sacrum area may be repositioned while the pressure sensors 208 corresponding to the head region and the shoulder area may be fixed.

In one example, the bottom layer 206 further includes one or more planks 210-1 and 210-2 as the movable part of the pressure sensing sheet 102 to allow adjustment of the pressure sensors 208. The one or more planks 210-1, and 210-2 are hereinafter collectively referred to as planks 210 and individually as plank 210. The planks 210 may be slidable across a predefined length of the bottom layer 206 to adjust a location of the plurality of pressure sensors 208 positioned on the planks 210. In one embodiment, the planks 210 may further include one or more orifices (not shown in the Figure) to receive a plurality of pipes 212-1 and 212-2, hereinafter, collectively referred to as pipes 212, disposed on the bottom layer 206. The pipes 212 acts as the guides for the planks 210 such that the planks 210 may be slided over and along the plurality of pipes 212 to allow adjustment of the pressure sensors 208. For example, as shown in FIG. 2C, the planks 210-1 and 210-2 include orifices 214-1 and 214-2 respectively to receive the pipes 212. The planks 210-1 and 210-2 may thus slide over and around the pipes 212 to allow adjustment of the planks 210 along the pipes 212 to adjust the location of the plurality of sensors 208 positioned on the planks 210. The bottom layer 206 may further include one or more attaching members 216 on either side of the bottom layer 206, to fix the planks 210 to the bottom layer 206 after sliding the plank 210 to a certain position. In one example, a first side of the attaching member 216 may be attachable to the bottom layer 206 and a second side of the attaching member 216 may be detachably attachable to the plank 210 after sliding the plank 210 to a certain position. In on example, the attaching member 216 may be a Velcro, button, adhesive tapes, etc. In one example, the one or more planks 210 may be slided manually across the predefined length to adjust the location of the plurality of sensors 208. The one or more planks 210 may include one or more handles attachable to the bottom layer 206. In one example, the handles may pass through the pipes 212-1 and 212-2 to protrude outside of the bottom layer 206 to allow the planks 210 to be moved along with the pressure sensors 208.

For example, as shown in FIG. 2D, the planks 210 includes one or more handles 218 and 220 passing through the pipes 212-1 and 212-2 such that the plank 210-1 may be slided in a direction X using the handles 218 to adjust the position of the plurality of sensors 208. This facilitate adjusting one or more of the plurality of sensors 208 to be adjusted depending on height, weight, age and size of the user's body. Thus, users with different height, weight, size, and age may use the multiside tilt device 100, thereby providing a flexible multiside tilt device. Further, adjusting the already available pressure sensors would require only a few pressure sensors to monitor the pressure of the entire body of the user. Using only a few pressure sensors thus reduces the complexity and cost involved in manufacturing the multiside tilt device 100, thereby providing a simple and cost-effective tilting device.

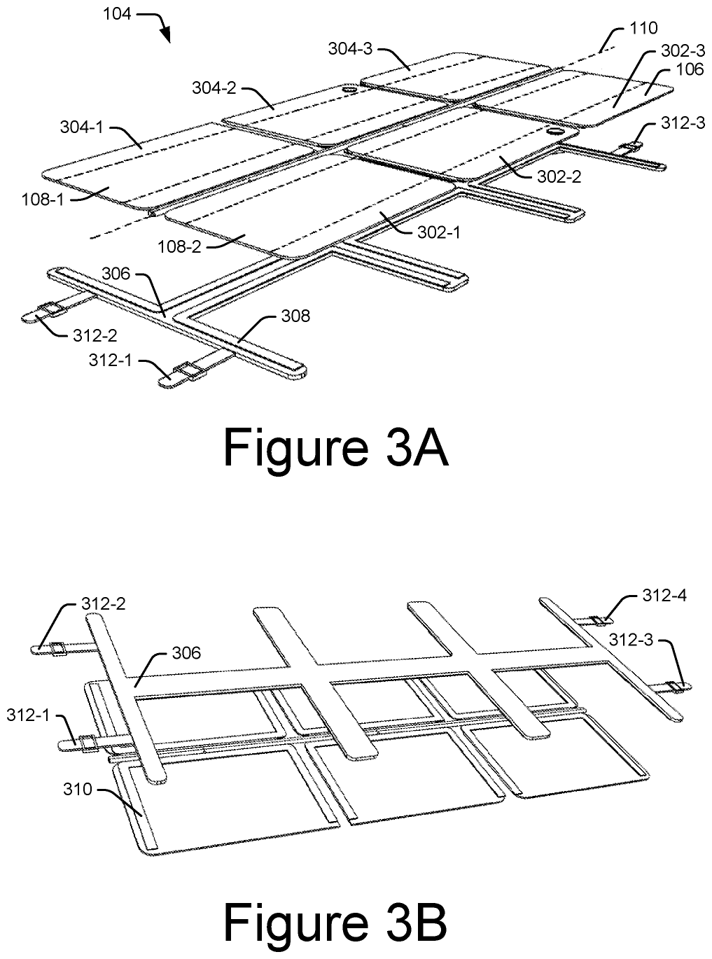

FIG. 3A illustrates the tilting unit 104 of the multiside tilt device 100, in accordance with an implementation of the present subject matter. In one example, the tilting unit 104 may facilitate tilting of the user's body in a lateral direction to relieve the pressure build-up occurring at the pressure build-up areas. In one example, the pressure build-up area may correspond to bony prominence areas. Examples of the bony prominence areas may include, but not limited to, occiput (back of head), scapula (shoulder blades), sacrum (tailbone area) and heels.

The tilting unit 104 includes the inflatable base 106 having the first section 108-1 and the second section 108-2 disposed adjacent to each other on either side of the median axis 110 of the tilting unit. In one example, in the resting position, the user's spinal curve lies in the region corresponding to the median axis. The median axis 110 divides the user's body into two symmetrical halves making it easy to position the plurality of sensors on selective areas. The median axis 110 may thus be presumed to be dividing the user's body into two symmetrical halves such that the plurality of sensors may be equally positioned in the predetermined areas.

The first section 108-1 and the second section 108-2 are pneumatically separated from each other to be independently inflated or deflated in response to the pressure signal. Each section 108 includes one or more cells pneumatically connected to each other to be collectively inflated or deflated in response to the pressure signal. For example, the second section 108-2 includes the cells 112-1, 112-2, and 112-3, that may be pneumatically connected to each other to be collectively inflated in response to the pressure signal. For the sake of brevity and consistency, the cells 112-1, 112-2, and 112-3, as illustrated in FIG. 1B are hereinafter interchangeably referred to as 302-1, 302-2, and 302-3. The one or more cells 302-1, 302-2 and 302-3 corresponding to the second section 108-2 are hereinafter referred to as one or more cells 302 and individually as cell 302. Similarly, the first section 108-1 includes one or more cells 304-1, 304-2, and 304-3 that may be pneumatically connected to each other to be collectively inflated or deflated in response to the pressure signal. The one or more cells 304-1, 304-2, and 304-3 corresponding to the first section 108-1 are hereinafter referred to as one or more cells 304 and individually as cell 304. In one example, the cells 112, 304, of both the sections 108 may be individually attached or detached to the inflatable base 106, thereby making the tilting unit modular. This overcomes the drawback of most of the conventional air mattresses used for pressure sore prevention wherein damage to any one of the cell make the mattress unit unusable. Since, each cell 112, 304, may be individually attached or detached, the replacement process is made easy and user-friendly. Replacing single units of cells 112, 304, may also not require any expertise and thus, a specialized medical professional may not be required to change or operate the tilting unit.

In one example, each cell 112, 304, is inflated to form an inflated structure having a peak in the center and a trough on either side. When one of the section 108 is inflated, a part of user's body resting over the section 108, is in contact with the peak of each cell 112, 304, while other parts of the user's body resting over the section 108, and corresponding to the trough, are not in contact with the multiside tilt device 100, to release pressure build-up.

The tilting unit 104 may further include a support base 306 to support the inflatable base 106 and provide strength to the tilting unit 104. The support base 306 may include a receiving portion 308 to receive a protruding position (not shown in this Figure) of the inflatable base 106. The receiving portion 308 of the support base 306 and the protruding portion of the inflatable base 106 may be attached or detached to each other, forming a male-female connection. For example, as shown in FIG. 3B, the protruding portion 310 of the inflatable base 106 may be received by the receiving portion 308 of the support base. In one example, the protruding portion 310 and receiving portion 308 may be made from any stickable material, such as Velcro or any tough fabric material resistant to wear and tear which is flexible, rollable and bendable. In another example, the inflatable base 106 and the support base 306 may be attached using buttons such that the male part of the buttons may correspond to the protruding portion 310 and the female part of the buttons may correspond to the receiving portion 308.

The support base 306 may further include a plurality of straps 312-1, 312-2, 312-3 and 312-4, hereinafter collectively referred to as straps 312, extending laterally outwards from the support base to tie the tilting unit 104 to the user's bed or any other similar resting surface. Tying the support base 306 to the user's bed helps the pressure sensing sheet 102 and the tilting unit 104 to remain in a fixed position and prevents the user from sliding.

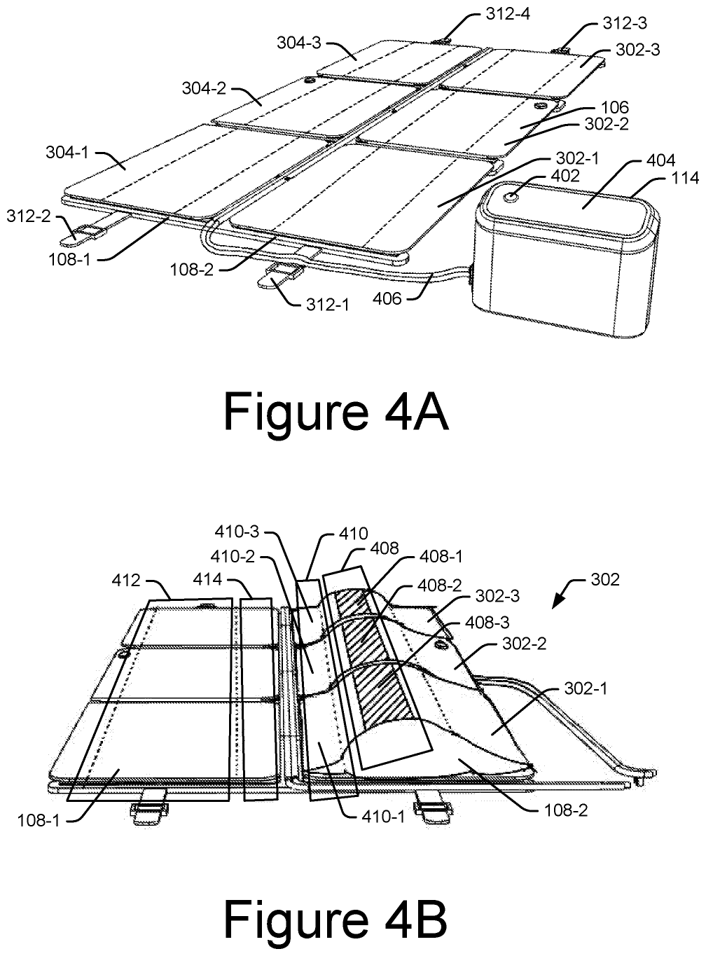

FIG. 4A illustrates the tilting unit 104 and the control unit 114 of the multiside tilt device 100, in accordance with an implementation of the present subject matter. The inflation and deflation of the first section 108-1 and the second section 108-2 is controlled by the control unit 114.

In one example, the control unit 114 may include a power button 402 to operate the control unit 114 in an ON state and an OFF state. Further, the control unit 114 may include input/output (I/O) interface(s) 404, memory (not shown in the figure), and processor(s) (not shown in the Figure). The I/O interface(s) 404 may allow the user to interact with the control unit 114 and view the pressure signal/pressure image received from the plurality of sensors 208 on a real-time basis. The memory may store threshold pressure values and instantaneous values of the pressure signals received form the pressure sensors. The memory may include any non-transitory computer-readable medium including, for example, volatile memory such as RAM, or non-volatile memory such as EPROM, flash memory, and the like. The control unit 114 further includes an audio/visual unit (not shown in the Figure) to provide indication to the user to initiate an inflation process.

The control unit 114 may further include a fluid directing unit (not shown in the Figure) to direct fluid, such as air, in and out of the first section 108-1 and the second section 108-2 of the inflatable base 106. The fluid directing unit may include a fluid pump, a vacuum pump, and one or more flow regulators. The first section 108-1 and the second section 108-2 of the inflatable base 106 may be inflated by pumping fluid through the fluid pump into any one of the first section 108-1 and the second section 108-2. The vacuum pump creates a vacuum in the first section 108-1 and the second section 108-2 to deflate the fluid from any one of the first section 108-1 and the second section 10-2. The one or more flow regulators are coupled to the fluid pump and the vacuum pump to transfer fluid to and from the control unit 114 and both the sections of the inflatable base 106. The one or more air flow regulators may include one or more controllable valves to enable or disable fluid communication of the vacuum pump and the fluid pump with the sections 108 of the inflatable base 106. In one example, the one or more controllable valves may be solenoid valves.

The control unit 114 may further include a distribution channel 406 in fluid communication with the first section 108-1 and the second section 108-2 of the inflatable base 106. The distribution channel 406 acts as a passage for fluid flowing between the fluid directing unit and the sections 108 of the inflatable base 106. In one example, the distribution channel 406 may include tubular pipes placed on the median axis and extending across the one or more cells of the sections 108.

The control unit 114 may receive the pressure signal from the plurality of sensors 208 indicating the pressure build-up at the pressure build-up area. The value of the pressure signal received is then compared with a threshold pressure value. If the value of the pressure signal at the pressure build-up area is greater than the threshold pressure value, the control unit 114 may determine that the user's body has to be tilted by a predetermined turning angle to relieve the pressure build-up. In one example, the control unit 114 may initiate an inflation process to inflate the section 108 of the tilting unit 104 corresponding to the pressure build-up area to laterally turn a user's body by the predetermined tilting angle. In another example, the control unit 114 provides an indication to the user to initiate an inflation process to inflate a section 108 of the tilting unit 104 corresponding to the pressure build-up area to laterally turn a user's body by the predetermined turning angle. In one example, the predetermined turning angle may be any angle in the range of 0 degree to 30 degrees. Subsequently, the section 108 of the inflatable base 106 corresponding to a pressure build-up area may be manually or automatically inflated. If the value of the pressure signal at the pressure build-up area is less than the threshold pressure value, the control unit may continue to monitor the pressure signal from the plurality of sensors.

FIG. 4B illustrates the one or more cells 302-1, 302-2, and 302-3 of the second section 108-2 in an inflated state. Each cell 302 of the second section 108-2 is inflated to form an inflated structure having a peak region. For example, the cells 302-1, 302-2, and 302-3 may be inflated to form peak regions 408-1, 408-2, and 408-3, respectively. The peak region 408-1, 408-2, and 408-3 are hereinafter collectively referred to as peak region 408. Further, as discussed earlier, a user may rest on the device 100 such that the user's spinal curve lies in the region corresponding to the median axis 110. In the inflated state, the region of the first section 108-1 proximate to the median axis 110 may be referred to as the trough region 410. Each cell 302-1, 302-2, and 302-3 may form its respective troughs, i.e., 410-1, 410-2, and 410-3, collectively referred to as trough region 410. Similarly, cells 304-1, 304-2, and 304-3 may be inflated to form its respective peaks (not shown in this figure) and troughs (not shown in this figure) in the regions 412 and 414, respectively. Thus, when a user is resting on the device 100, parts of the user's body corresponding to the peak region 408 may be supported by the inflatable base 106 while the parts of the user's body corresponding to the trough region 410 may be lifted. In one example, the parts of the user's body corresponding to only the trough region 410 of the inflated section 108-2 may be lifted. In another example, the parts of the user's body corresponding to the trough region 410 of the inflated section 108-2 and the trough region 414 of the deflated section 108-1 may be lifted.

This allows air to flow in the trough regions and reduce the moisture build-up in the outer layer of the skin, thereby reducing the probability of bed sores.

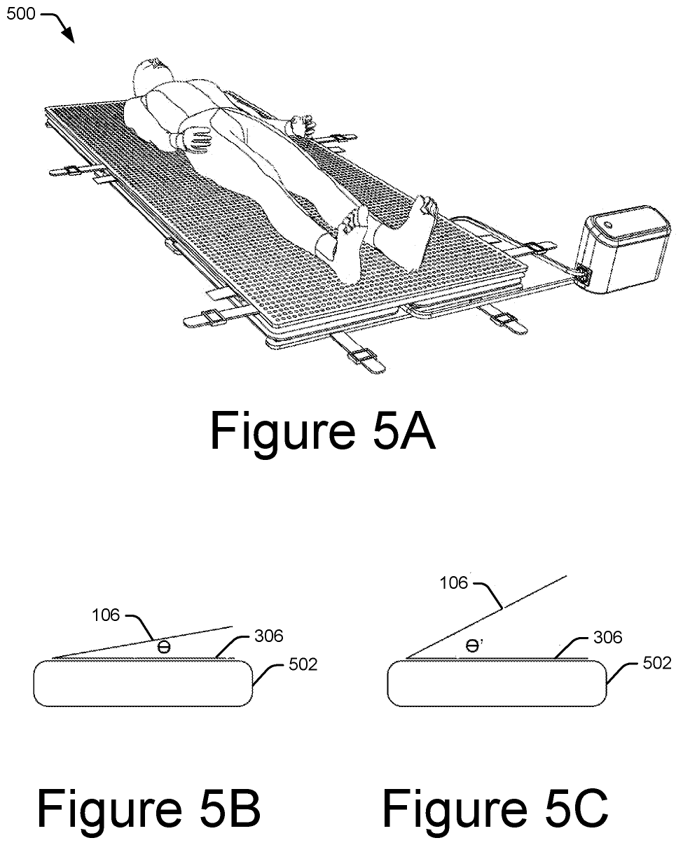

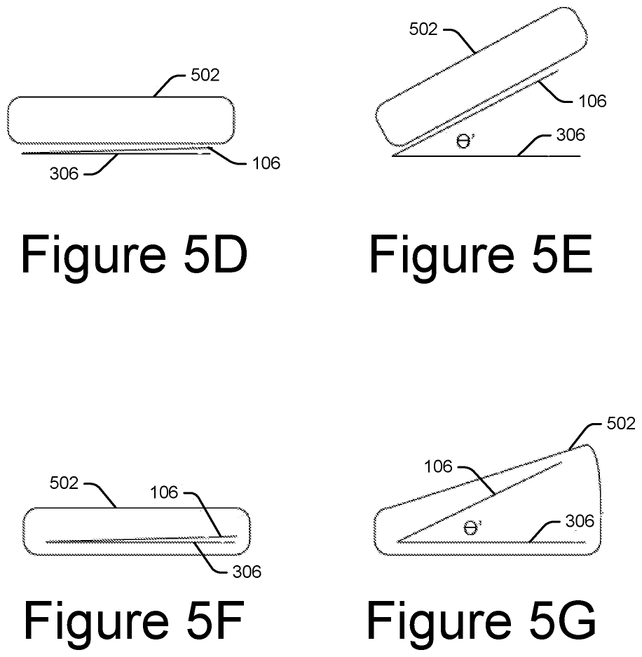

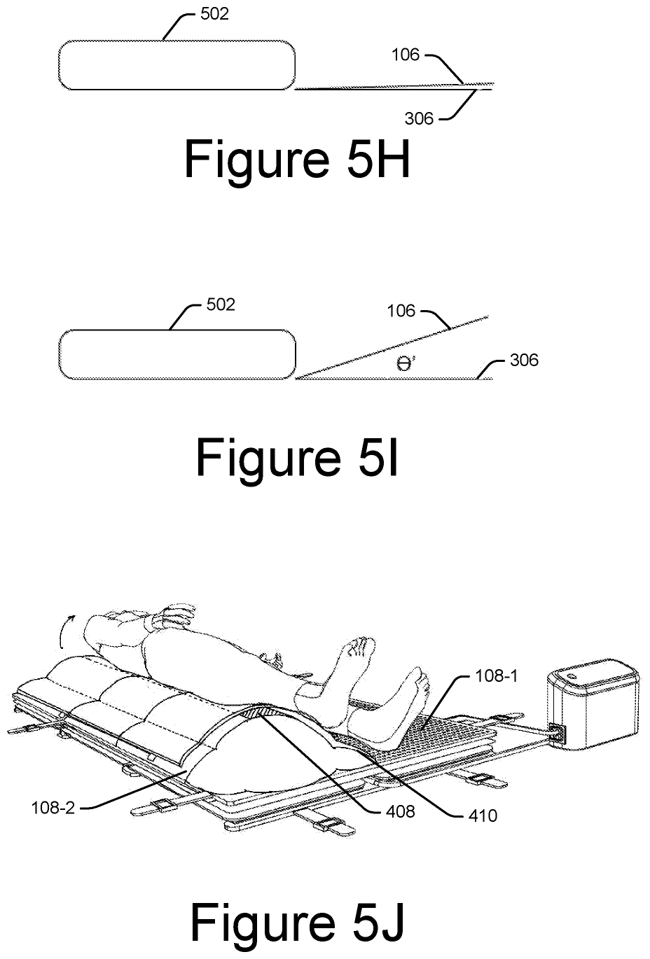

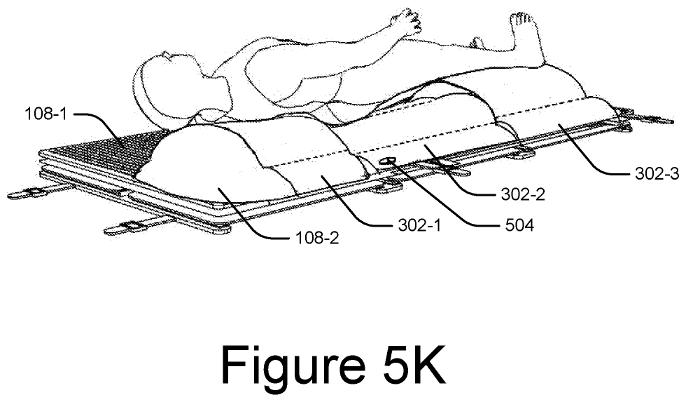

FIG. 5A-5B illustrates different stages of operation of the multiside tilt device, in accordance with an implementation of the present subject matter.

In operation, to reposition a user to relieve him from pressure build-up at a pressure build-up area, the user may be resting on the multiside tilt device 100 such that the spinal curve of the user lies on the median axis 110 of the multiside tilt device 100. In one example, the tilting unit 104 may be positioned above a resting surface. For example, FIGS. 5B-5C illustrate example implementations of the tilting unit 104 when placed above a resting surface 502 in a normal and an inflated position. In another embodiment, the tilting unit 104 may be placed below a resting surface 502. For example, FIGS. 5D-5E illustrates example implementations of the tilting unit 104 when placed below resting surface in a normal and an inflated position. In yet another example, the tilting unit 104 may be placed inside the resting surface 502. In this example, the resting surface 502 may be made of any material that is elastic or flexible or composite or combination of them. For example, FIGS. 5F-5G illustrates example implementations of the tilting unit 104 when it is placed inside the resting surface 502 in a normal and inflated position. In yet another example, the tilting unit 104 may be placed sideways to the resting surface 502. For example, FIGS. 5H-5I illustrates example implementations of the tilting unit 104 when it is placed sideways to the resting surface 502 in a normal and an inflated position. The pressure sensing sheet (not shown in FIGS. 5B-5I) may always be placed above the resting surface, such the resting surface 502.

In operation, as the user is resting on the multiside tilt device 100, the control unit 114 monitors pressure exerted by the user's body at the pressure build-up areas. In one example, the pressure build-up areas may correspond to bony prominence areas. Examples of bony prominence areas may include, but are not limited to, occiput (back of head), scapula (shoulder blades), sacrum (tailbone area) and heels. In one example, the pressure sensors 208 may periodically share pressure signals indicating the pressure exerted by the user on the device 100. In another example, the pressure sensors 208 may generate the pressure signal only upon detecting pressure build-up in the pressure build-up area. Upon receiving the pressure signal, the control unit 114 may compare the value of the received pressure signal with the threshold pressure value. If the value of the pressure signal at the pressure build-up area is greater than the threshold pressure value, the control unit 114 may determine that the user's body may be tilted by a predetermined turning angle. For example, as illustrated in FIG. 5J, the one or more cells 302 have been inflated in response to a given pressure signal. Each of the cells 302 forms an inflated structure having the peak 408 in the center and the trough 410 on either side such that a part of user's body resting over the section 108-2, is in contact with the peak of each cell, while other parts of the user's body resting over the section, and corresponding to the trough, are not in contact with the multiside tilt device 100.

This intentional design lifts the body by supporting the side areas of the body, resulting in leaving contact of the bony prominence areas like sacrum off the surface. For example, as illustrated in FIG. 5J, parts of the user's body corresponding to the peak region 408 remains in contact with the section while the other parts of the body corresponding to the trough region 410 may be lifted and may not be in contact with the section. The body is thus lifted from the sides and the central region remains untouched with the surface resulting in no pressure build-up in the central areas like sacrum at the same time providing aeration to those unexposed regions. This would result in laterally turning the user resting on top of the resting surface, which in turn shifts and redistribute the pressure acting on the user's body.

In one example, the one or more cells 112, 304 corresponding to the sections 108 may include at least one cell having a release valve such that a manual input from the user independently deflates the at least one cell. For example, as illustrated in FIG. 5K, the cell 302-2 may include a release valve 504 that may be actuated based on a manual input by the user to release the fluid. Examples of the manual input may include pressing the valve and opening the valve. As a result, the cell 302-2 is deflated while the other cells 302-1 and 302-3 remain inflated. Thus, the cell 302-2 may gradually deflate releasing the support from region corresponding to cell 302-2. In this case, the sacrum area pressure is relieved and the body of the user is supported by the other two cells, i.e., 302-1 and 302-3. This is helpful when the patient has high likelihood of developing a pressure ulcer or have already developed a pressure ulcer in a given area, such as the sacrum. In that case the given area needs to be exposed to the atmosphere and the pressure acting in that area should be the least as possible. This intervention of releasing air through release valve 504 will help in accomplishing that in the lateral turned position.

Although examples for the present subject matter have been described in language specific to structural features and/or methods, it should be understood that the appended claims are not limited to the specific features or methods described. Rather, the specific features and methods are disclosed and explained as examples of the present subject matter.

1. A multiside tilt device comprising:
a pressure sensing sheet to support a user in a resting position, wherein the pressure sensing sheet comprises a plurality of pressure sensors, and wherein each of the plurality of pressure sensors is to:
detect pressure exerted by the user's body in a pressure build-up area around the pressure sensor; and
generate a pressure signal indicating a pressure build-up at the pressure build-up area;
a tilting unit positioned beneath the pressure sensing sheet, wherein the tilting unit comprises
an inflatable base having a first section and a second section disposed adjacent to each other on either side of a median axis of the tilting unit, wherein in the resting position the user's spinal curve lies in region corresponding to the median axis, wherein both the sections are pneumatically separated from each other to be independently inflated or deflated in response to the pressure signal, and wherein each section comprises:
one or more cells pneumatically connected to each other to be collectively inflated in response to the pressure signal, wherein each cell is inflated to form an inflated structure having a peak in the centre and a trough on either sides, wherein a part of the user's body resting over the section, is in contact with the peak of each cell, while other parts of the user's body resting over the section, and corresponding to the trough, are not in contact with the multiside tilt device to release the pressure build-up.

2. The multiside tilt device as claimed in claim 1, wherein at least one cell from the one or more cells comprises a release valve to independently deflate the at least one cell in response to a manual input from the user.

3. The multiside tilt device as claimed in claim 1, wherein the first section is to support a right side of the user's body and the second section is to support a left side of the user's body.

4. The multiside tilt device as claimed in claim 1, wherein the pressure sensing sheet comprises:
a top layer having perforations for air passage to allow air to flow through the top layer and;
a bottom layer coupled to the top layer to form a sealed sheet, wherein the bottom layer comprises
one or more planks slidable across a predefined length of the bottom layer; and
the plurality of pressure sensors positioned on the one or more planks, wherein the one or more planks are slidable across the predefined length to adjust the location of the plurality of pressure sensors in the pressure sensing sheet.

5. The multiside tilt device as claimed in claim 1, wherein the tilting unit comprises:
a support base comprising:
a receiving portion to receive a protruding portion of the inflatable base to attach or detach the inflatable base from the support base; and
a plurality of straps extending laterally outwards from the support base to tie the tilting unit to the user's bed.

6. The multiside tilt device as claimed in claim 1, comprises a control unit to:
receive the pressure signal indicating the pressure build-up at the pressure build-up area;
compare a value of the pressure signal with a threshold pressure value; and
for the value of the pressure signal at the pressure build-up area being greater than the threshold pressure value, determine that the user's body has to be tilted by a predetermined turning angle to relieve the pressure build up.

7. The multiside tilt device as claimed in claim 6, wherein the control unit is to:
initiate an inflation process to inflate a section of the tilting unit corresponding to the pressure build up area to laterally turn the user's body by the predetermined turning angle.

8. The multiside tilt device as claimed in claim 6, wherein the control unit is to:
provide an indication to the user to initiate an inflation process to inflate a section of the tilting unit corresponding to the pressure build up area to laterally turn the user's body by the predetermined turning angle.

9. The multiside tilt device as claimed in claim 6, wherein the predetermined turning angle is in a range of 0 degree to 30 degrees.

10. The multiside tilt device as claimed in claim 6, wherein the control unit comprises:
a fluid directing unit comprising:
a fluid pump to pump a fluid into the first and the second section of the inflatable base to inflate;
a vacuum pump to deflate the fluid from the first section and the second section of the inflatable base; and
one or more air flow regulators coupled to the fluid pump and the vacuum pump to transfer fluid to and from the control unit and both the sections of the inflatable base, wherein the one or more air flow regulators comprises one or more controllable valves for directing the fluid flow from the fluid pump and the vacuum pump to both the sections of the inflatable base.

11. The multiside tilt device as claimed in claim 9, further comprising a distribution channel in fluid communication with the first section and the second section of the inflatable base; wherein the distribution channel acts as a passage for fluid flowing between the fluid directing unit and both the sections of the inflatable base.
